(19) United States
(12) Patent Application Publication
(10) Pub. No.: US 2005/0255230 A1
(43) Pub. Date: Nov. 17, 2005

(54) Method of manufacturing a covered stent

(76) Inventors: Claude O. Clerc, Marlborough, MA (US); Kathleen M. Miller, Shrewsbury, MA (US)

Correspondence Address: KENYON & KENYON, 1500 K STREET NW, SUITE 700, WASHINGTON, DC, 20005, US

(21) Appl. No.: 10/846,706

(22) Filed: May 17, 2004

(51) Int. Cl. A61L 2/00; B05D 3/00

(52) U.S. Cl. 427/2.1

(57) ABSTRACT

A method of manufacturing a covered stent having a sufficiently thick covering to retain a therapeutically effective amount of a therapeutic agent. The covering is applied to the entire outer surface of the stent to provide sufficient volume for retention of the therapeutic agent. In certain embodiments, the stent has a plurality of openings that are covered by the covering. The invention is particularly suited for certain applications, such as for the manufacture of non-vascular stents.

### FIELD OF THE INVENTION

The present invention relates to a method of manufacturing a covered stent by forming a covering on the stent with a viscous mixture.

### BACKGROUND OF THE INVENTION

Stents, such as non-vascular stents, are often used to open or maintain patency of constricted lumens or to provide drainage through obstructed lumens of tubular organs or tissue. Such lumens can become constricted or obstructed as a results of injury or disease. For example, in esophageal cancer, the pathway through the esophagus often narrows as a result of the tumor spreading outward from the inside lining of the esophagus. An esophageal stent may be placed in the esophagus to open or maintain open the esophagus to allow for the intake or to decrease the discomfort associated with the intake of food and water. In obstructive jaundice, the bile ducts may be obstructed as a result of inflammation,.cholangitis, gallstones, or cancer of the pancreas or the common bile duct, thereby causing an excessive accumulation of bilirubin in the body. A biliary stent may be placed in the bile duct to allow the bile to drain through the bile duct into the small intestine.

A non-vascular stent that is configured to be placed at a diseased site, such as a cancerous site, generally has a thin film around the center of the outer surface and has exposed ends. The thin central film prevents penetration of the tumor into the stent lumen. It may be desirable, however, to also provide localized delivery of a therapeutic agent to the diseased site. Compared to systemic drug administration, such localized drug delivery minimizes unwanted effects on parts of the body which are not to be treated and allows for the delivery of higher concentrations of therapeutic agent to the afflicted part of the body. The current thin films around the center of the outer surface of existing non-vascular stents, however, may be unsuitable in some cases to carry the amount of drug needed for therapeutically effective drug delivery to the diseased non-vascular site. Likewise, existing conformal covering processes used in vascular stents, which result in only the stent struts being coated, are also unsuitable to carry the amount of drug needed for delivery to a non-vascular target site.

Accordingly, there is a need in the art for a method of manufacturing a covered stent, particularly a covered non-vascular stent, which results in inhibition of tumor growth as well as hyperplastic or granulation tissue growth into the stent or in the proximity of the stent. There is also a need in the art for a method of manufacturing a covered stent, particularly a covered non-vascular stent that also allows for a greater amount of drug to be delivered by the stent.

### SUMMARY OF THE INVENTION

The present invention provides a method of manufacturing a covered stent, particularly a covered non-vascular stent, comprising a plurality of segments defining a plurality of openings therebetween. The method includes preparing a viscous mixture of a polymer, a solvent, and a therapeutic agent and applying the viscous mixture to the stent to form a covering that covers both the plurality of segments and the plurality of openings. The method further comprises allowing the solvent to evaporate.

The present invention also provides a method of manufacturing a covered stent, particularly a covered non-vascular stent, comprising a hollow tube having a continuous outer surface. The method includes preparing a viscous mixture of a polymer, a solvent, and a therapeutic agent and applying the viscous mixture to the stent to form a covering on the entire continuous outer surface of the stent. The method further comprises allowing the solvent to evaporate.

The present invention moreover provides a method of manufacturing a covered stent, particularly a covered non-vascular stent, having an outer surface. The method includes preparing a viscous mixture of a polymer, a solvent, and a therapeutic agent wherein the mixture has a viscosity of between about 110 centipoise and about 190 centipoise. The method further comprises applying the viscous mixture to the entire outer surface of the stent and allowing the solvent to evaporate.

The present invention additionally provides a method of treating a non-vascular target site. The method includes providing a non-vascular stent having an outer surface that is entirely covered with a covering comprising a polymer and a therapeutic agent. The method further comprises delivering the non-vascular stent to the non-vascular target site and allowing the therapeutic agent to be released into the non-vascular target site to treat the non-vascular target site.

### BRIEF DESCRIPTION OF THE DRAWINGS

The present invention will become more fully understood from the detailed description given hereinbelow and the accompanying drawings which are given by way of illustration only and wherein:

FIG. 1 is a perspective view of a covered stent according to the present invention.

FIG. 2 is a side view of a covered stent according to the present invention.

### DETAILED DESCRIPTION

The present invention provides a method of manufacturing a covered stent, particularly a covered non-vascular stent, that includes forming a covering on the entire outer surface of the stent. The covering effectively serves as a reservoir for retaining a therapeutic agent on the stent. Because the entire outer surface of the stent is covered, the reservoir extends across the entire outer surface of the stent. The covering is formed by applying to the outer surface of the stent, a viscous mixture comprising a polymer, a solvent, and a therapeutic agent (and any combinations and multiples thereof) and then allowing the solvent to evaporate. The viscosity of the mixture and the resulting thickness of the covering is sufficient to retain a therapeutically effective amount of a therapeutic agent for delivery to a non-vascular site. The therapeutically effective amount is the amount of the therapeutic agent that is effective in producing the desired biological effect of the agent. The method of manufacturing a stent according to the present invention is different from conventional coating processes in several respects. Specifically, the method involves covering the stent with a viscous mixture such that the entire outer surface of the stent is covered with the viscous mixture. After the solvent is evaporated from the viscous mixture, a thick continuous polymer covering remains and the therapeutic agent is uniformly distributed therein. This covering process is distinct from conformal coating processes where only the struts of the stent are coated. Further, because the therapeutic agent is distributed through the whole thickness of the polymeric coating including in the covering spanning the struts (including openings between struts), the stent carries a larger quantity of therapeutic agent than if the therapeutic agent was subsequently added as part of a thin film over the stent or if the therapeutic agent was applied to the stent by a conformal coating process.

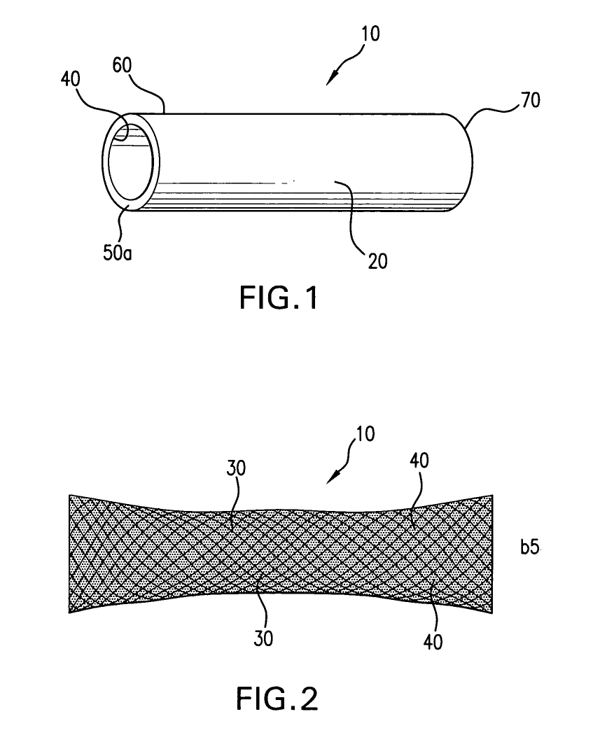

Referring to FIG. 1, a covered stent 10 manufactured according to a method of the present invention, has a proximal end 60 and a distal end 70 and may have a hollow tubular shape with a continuous outer surface 20 such that outer surface 20 defines no openings or gaps. According to a method of the present invention, the entire continuous outer surface 20 is covered with a polymeric covering containing a therapeutic agent. Referring to FIG. 2, a stent 10 manufactured according to the present invention may alternatively have a discontinuous outer surface comprising a plurality of segments or struts 30 defining a plurality of openings 40 therebetween. According to a method of the present invention, both the plurality of segments 30 and the plurality of openings 40 are covered with a covering containing a therapeutic agent. Such a method is in contrast to the conformal coating process used in vascular stents where openings defined by the stent are not occluded by a covering as such a covering would diminish the vascular tissue uptake of required nutrients from the blood supply.

Although a feature of the present invention is covering the entire outer surface of a stent with a viscous mixture resulting in a polymeric covering, other surfaces of the stent may also be covered. For example, referring back to FIG. 1, the inner surface 40, edge surface 50a at proximal end 60 of stent 10, and the edge surface (not shown) at the distal end 70 may also be covered with the covering to provide even greater surface area for the therapeutic agent. Alternatively, proximal end 60, distal end 70, or both may be left uncovered to improve stent anchoring and reduce stent migration. In this alternative embodiment, however, the the outer surface of the stent between the proximal and distal ends 60 and 70 is entirely covered.

It should be understood that the stents depicted in FIGS. 1 and 2 are merely exemplary, and the present invention contemplates manufacture of a stent having any shape or configuration. For example, the stent could have the shape of a coil stent, spiral stent, zigzag stent, or a mesh stent (including a patterned stent such as a braided, woven, or knitted stent). The stents according to the present invention can be configured to be deployed in any non-vascular target site such as, for example, the gastrointestinal tract such as the bile duct, esophagus, pancreas, duodenum, or colon; the respiratory tract, such as the trachea, larynx, or bronchial tubes; and the urinary/urological tract, such as the prostate, ureter or urethra. Such stents can be self-expanding or balloon expandable and can be fabricated of any biocompatible material such as metallic, non-metallic, or shape memory materials. The size of the stent will depend on its specific application. Non-limiting examples of the sizes of stents (in a deployed state) manufactured according to the present invention are listed in Table I.

TABLE I

| | Outer Diameter (mm) | Stent Length (mm)* |
|---|---|---|
| Biliary Stent | 8-10 | 30-70 |
| Esophageal Stent | 18-23 | 70-150 |
| Tracheo-Bronchial | 8-23 | 10-80 |
| Stent | | |
| Colonic Stent | 20-25 | 60-120 |
| Prostatic/Urethral | 12-14 | 10-50 |
| Stent | | |
| Ureteral Stent | 5-10 | 20-100 |

In order to form a covering of sufficient thickness around the entire outer surface of the stent, the methods of the present invention comprise preparing a viscous mixture comprising a polymer, a solvent, and a therapeutic agent, applying the viscous mixture to the stent to form the covering, and then allowing the solvent to evaporate. This step can be repeated until a covering of desirable thickness has been obtained. Preferably, the viscosity of the mixture is between about 50 and 500 centipoise (cP) (particularly if the polymer is silicone). More preferably the viscosity is between 110 cP and 190 cP. Preferably, the weight percent solid of polymer in the viscous mixture is between about 5 to 25%, more preferably between 20% and about 25% (particularly if the polymer is silicone), and even more preferably between 22% and 23.5%. The weight percent of therapeutic agent in the viscous mixture is between about 0.1% and about 6%. The percent of therapeutic agent in the covering after the solvent has evaporated is between about 0.4% and about 50%.

Preparing a mixture having a viscosity sufficient to form a covering that can retain a therapeutically effective amount of a therapeutic agent involves, for example, choosing the appropriate polymer and solvent combination, the appropriate amount of solvent, and/or the appropriate weight percentage of polymer and solvent. Examples of suitable combinations of polymers and solvents are listed in Table II below.

TABLE II

| | | Polymer |
|---|---|---|
| Polymer | Solvent | Weight % |
| silicone | xylene, hexane | 20-25 |
| polyuretane | DMAC, chloroform, methylene | 10-20 |
| | chloride | |
| styrene-isobutylene-styrene | toluene, ethyl acetate, methylene | 10-20 |
| (TRANSLUTE ™) | chloride | |

In a preferred embodiment, the polymer is a biomedical grade elastomer such as silicone or polyurethane. In the case of silicone, the viscous mixture comprises 20% to 25% weight silicone, such as Nusil MED-4820 (NuSil Technology, Santa Barbara, Calif.), in xylene and between about 0.1% and about 6% weight of therapeutic agent. Such a combination would result in a final drug loading of between about 0.4% to about 23% weight of the dry covering (i.e. after the solvent has evaporated) with 77% to 99.6% weight of the covering comprising the polymer or the polymer plus an opacifying agent (discussed below). In the case of polyurethane, such as Chronflex AR (Cardiotech International, Woburn, Mass.), the viscous mixture comprises between about 10% and 15% weight polyurethane in dymethylacetamide (DMAC) and between about 0.1% to about 5% weight of therapeutic agent. Such a combination would result in a final drug loading of between about 0.7% to about 33% weight of the dry covering (i.e. after the solvent has evaporated) with 67% to 99.3% weight of the covering comprising the polymer or the polymer plus an opacifying agent (discussed below).

Of course, the above-listed polymer and solvent combinations are merely exemplary and other types of polymers and solvents that result in a sufficiently viscous mixture will be readily known to one in the art and are therefore within the scope of the present invention.

With respect to other types of polymers that are suitable for use according to the present invention, such other polymers can be biodegradable or non-biodegradable. Preferably, the polymer is thermoplastic, elastomeric, and/or bioresorbable. Non-limiting examples of suitable non-biodegradable suitable polymers includes polyolefins such as metallocene catalyzed polyethylenes, polypropylenes, and polybutylenes and copolymers thereof; vinyl aromatic polymers such as polystyrene; vinyl aromatic copolymers such as styrene-isobutylene copolymers including styrene-isobutylene-styrene (preferably TRANSLUTE™ manufactured by Boston Scientific) and butadiene-styrene copolymers or other block polymers; ethylenic copolymers such as ethylene vinyl acetate (EVA), ethylene-methacrylic acid and ethylene-acrylic acid copolymers where some of the acid groups have been neutralized with either zinc or sodium ions (commonly known as ionomers); polyacetals; chloropolymers such as polyvinylchloride (PVC); fluoropolymers such as polytetrafluoroethylene (PTFE); polyesters such as polyethyleneterephthalate (PET); polyester-ethers; polyamides such as nylon 6 and nylon 6,6; polyamide ethers; polyethers; elastomers such as elastomeric polyurethanes and polyurethane copolymers (including silicone-polyurethane copolymers and polycarbonate-urethane polymers); silicones; polycarbonates; and mixtures and copolymers of any of the foregoing.

Non-limiting examples of suitable biodegradable polymers include polylactic acid, polyglycolic acid and copolymers and mixtures thereof such as poly(L-lactide) (PLLA), poly(D,L-lactide) (PLA); polyglycolic acid [polyglycolide (PGA)], poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-cotrimethylene carbonate) (PGA/PTMC), poly(D,L-lactide-co-caprolactone) (PLA/PCL), poly(glycolide-co-caprolactone) (PGA/PCL); polyethylene oxide (PEO), polydioxanone (PDS), polypropylene fumarate, poly(ethyl glutamate-co-glutamic acid), poly(tert-butyloxy-carbonylmethyl glutamate), polycaprolactone (PCL), polycaprolactone co-butylacrylate, polyhydroxybutyrate (PHBT) and copolymers of polyhydroxybutyrate, poly(phosphazene), poly(phosphate ester), poly(amino acid) and poly(hydroxy butyrate), polydepsipeptides, maleic anhydride copolymers, polyphosphazenes, polyininocarbonates, poly[(97.5% dimethyl-trimethylene carbonate)-co-(2.5% trimethylene carbonate)], cyanoacrylate, polyethylene oxide, hydroxypropylmethylcellulose, polysaccharides such as hyaluronic acid, chitosan and regenerate cellulose, and proteins such as gelatin and collagen, and mixtures and copolymers of any of the foregoing.

With respect to other types of solvents that are suitable for use in the present invention, non-limiting examples of suitable solvents include dimethylsulfoxide (DMSO), chloroform, acetone, water (buffered saline), xylene, methanol, ethanol, 1-propanol, tetrahydrofuran, 1-butanone, dimethylformamide, dimethylacetamide, cyclohexanone, ethyl acetate, methylene chloride, methylethylketone, propylene glycol monomethylether, isopropanol, isopropanol admixed with water, N-methyl pyrrolidinone, toluene, and combinations thereof.

The viscous mixture that is applied to a stent according to the present invention further comprises a therapeutic agent. The therapeutic agent may be any pharmaceutically acceptable agent. Exemplary therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, cladribine, 5-fluorouracil, methotrexate, doxorubicin, daunorubicin, cyclosporine, cisplatin, vinblastine, vincristine, epothilones, endostatin, trapidil, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as lisidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warafin sodium, Dicumarol, aspirin, prostaglandin inhibitors, platelet inhibitors and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogeneus vascoactive mechanisms; other hormones, sugars and lipids; compounds having a molecular weight of less than 100 kD; and any combinations of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, and siRNA; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12,BMP-13,BMP-14,BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase and combinations thereof. Non-limiting examples of angiogenic factors are acidic and basic fibroblast growth factors, vascular endothelial growth factor, epidermal growth factor, transforming growth factor a and β, platelet-derived endothelial growth factor, platelet-derived growth factor, tumor necrosis factor α, hepatocyte growth factor, and insulin like growth factor. A non-limiting example of a cell cycle inhibitor is a CD inhibitor. Non-limiting examples of anti-restenosis agents are p15, p16, p18, p19, p21, p27, p53, p57, Rb, nFkB and E2F decoys, thymidine kinase (“TK”) and combinations thereof and other agents useful for interfering with cell proliferation.

Exemplary cells include stem cells, progenitor cells, endothelial cells, adult cardiomyocytes, and smooth muscle cells. Cells can be of human origin (autologous or allogenic) or from an animal source (xenogenic), or genetically engineered.

Any of the therapeutic agents may be combined to the extent such combination is biologically compatible and chemically stable in the presence of the polymer and solvents used to manufacture the final stent covering.

Although the present invention has been described in terms of a therapeutic agent being contained within the covering, the present invention contemplates more than one therapeutic agent retained within the covering. For example, combinations of therapeutic agents can be added to the mixture depending, for example, on the particular use of the stent. For instance, if the stent is a non-vascular stent and if the non-vascular stent is to be deployed in the esophagus to treat esophageal cancer, than an anti-microbial agent and an anti-cancer agent could be included in the viscous mixture. The anti-microbial agent acts to prevent colonization of microbes on the stent, and the anti-cancer agent acts to prevent or inhibit tumor growth. If the non-vascular stent is to be deployed in an inflamed bile duct, for example, then an anti-microbial agent and an anti-inflammatory agent could be included in the viscous mixture. To the extent that the inflammation is a result of the microbes, such a combination relieves the inflammation and kills or controls the inflammation-causing microbes. Alternatively or in addition, different types of therapeutic agents for the same indication may be included in the viscous mixture. For example, different types of anti-microbial agents or different types of anti-restenosis agents may be included in the mixture. This may be particularly desirable if different types of therapeutic agents for the same indication have a synergistic and/or additive effect.

The amount of the therapeutic agent that is added to the mixture that covers the stent is a therapeutically effective amount according to the present invention. The exact amount of the therapeutic agent will depend, inter alia, on the particular therapeutic agent, the length of time during which the stent is intended to remain implanted, the rate at which the therapeutic agent is released from the covering, and the specific therapeutic needs of the target site. In the case of non-vascular stents, because many non-vascular target sites are larger than vascular target sites, the amount of therapeutic agent that is therapeutically effective for treating non-vascular sites may be greater than the amount of therapeutic agent that is therapeutically effective for treating vascular sites. Accordingly, the non-vascular stents to be delivered to these non-vascular sites must be capable of retaining a larger amount of therapeutic agent than a vascular stent. Because the methods of manufacturing according to the present invention include covering the entire outer surface of a stent with a thick covering, such a non-vascular stent provides sufficient volume for retention of a therapeutically effective amount of a therapeutic agent. In contrast, non-vascular stents having a thin film around the outer surface or vascular stents manufactured by a conformal covering process may not provide a sufficient volume for retention of a therapeutically effective amount of a therapeutic agent for delivery to a non-vascular target site.

The viscous mixture which is applied to a stent according to the present invention may also comprise a radiopacifying agent to facilitate viewing of the stent during insertion into the body and at any point while the stent is implanted. Non-limiting examples of radio-opacifying agents include bismuth subcarbonate, bismuth oxychloride, bismuth trioxide, barium sulfate, and metals such as tungsten, tantalum, gold, platinum and alloys and mixtures thereof. When present, the radio-opacifying agent is preferably present in an amount of from about 0.5% to about 90% by weight of the viscous mixture.

The viscous mixture that is applied to a stent according to methods of the present invention may be prepared by any method known to one in the art. For example, an initial polymer/solvent mixture can be prepared and then the therapeutic agent added to the polymer/solvent mixture. Alternatively, the polymer, solvent and therapeutic agent can be added simultaneously to form the mixture. Alternatively, a polymer/solvent “A” mixture and a separate therapeutic agent/solvent “B” mixture can be prepared and then mixed to form a final mixture. The polymer/solvent mixture may be a dispersion, suspension or a solution. The therapeutic agent may be dissolved in the polymer/solvent mixture to be in a true solution with the mixture, uniformly dispersed in fine sub-micron or micronized particles in the mixture, suspended in the mixture based on its solubility profile, or combined with micelle-forming compounds such as surfactants or adsorbed onto small carrier particles to create a suspension in the mixture. The mixture may comprise multiple polymers, multiple solvents, and/or multiple therapeutic agents. Multiple solvents may be employed when the polymer and therapeutic agent are not miscible or soluble in the same solvent. Complete dissolution of the therapeutic agent and the polymer may be preferred if it is desired to have the therapeutic agent distributed uniformly on the stent. In such an instance, a solvent is chosen for which the polymer exhibits a preferred solubility profile and another solvent is chosen for which the therapeutic agent exhibits a preferred solubility profile. The two solvents are combined under continuous mixing conditions and the resultant mixture is applied to the stent. Alternatively, upon combining the above two-solvent system under continuous mixing conditions, the therapeutic agent may form uniform sub-micron particulates. The resulting mixture is also then applied to the stent under continuous mixing conditions to yield a uniform distribution of therapeutic agent on the surfaces of the stent.

Once the viscous mixture is prepared, the mixture can be applied to the stent by any appropriate method known in the art. For example, the viscous mixture can be applied by dipping, spraying, rolling, brushing, electrostatic plating or spinning, vapor deposition, air spraying including atomized spray processes, and spray processes using an ultrasonic nozzle. The only limitation in the method of applying is the viscosity of the mixture to which the stent is exposed. Furthermore, the method of applying the viscous mixture must be capable of covering at least the entire outer surface of a non-vascular stent. In a preferred embodiment, the viscous mixture is applied to the stent by dipping the stent into the mixture and then allowing the solvent to evaporate. The viscous mixture may be applied to the stent any number of times to adjust the thickness of the covering.

After the stent is exposed to the viscous mixture, the stent is allowed to dry at ambient conditions or elevated temperatures with our without vacuum to allow the solvent to evaporate. Depending on the nature of the polymer, the polymer covering may then be cured by applying heat, light, and/or chemical agents to the polymer. To facilitate curing, a cross-linking or curing agent may be added to the viscous mixture prior to application onto the stent. Curing may also occur in situ by exposing the polymer covering containing the therapeutic agent to radiation such as ultraviolet radiation or laser light, heat, or by contacting the polymer covering with metabolic fluids such as water at the non-vascular site. Where, for example, polyurethane thermoplastic elastomers are used, solvent evaporation can occur at room temperature rendering the polymeric material useful for controlled drug release without further curing.

1 Additional layers of covering may also be deposited over the initial covering of the stent. Such additional layers may or may not contain additional therapeutic agent. For example, if it is desired to release a plurality of different therapeutic agents with different release kinetics, a plurality of different therapeutic agents with different release sequences, or the same therapeutic agent with a plurality of different release kinetics, additional covering layers with such therapeutic agents may be deposited over the initial covering of the stent. If, for example, it is desired to slow down the elution kinetics of the therapeutic agent contained within the initial covering, provide lubricity to the stent, or protect the initial covering from atmospheric degradation such as by oxidative or hydrolytic breakdown, then a top-covering or top-coverings without any therapeutic agent may be deposited over the initial covering. Alternatively or in addition, a pre-coat could be applied to the stent before application of the initial covering to enhance binding of the initial covering to the stent. The additional layers of covering, the topcoat, and/or the precoat may comprise the same or different polymeric compositions as the initial covering and such polymeric compositions may be chosen to provide different release characteristics of the therapeutic agent therein. For example, some compositions may result in relatively fast release while others may result in a relatively slower release profile. By appropriate selection and arrangement of the additional layers containing therapeutic agents, the solvents used in the process, the type of process used to apply the covering, and the drug to polymer ratio, the release profile of the different therapeutic agents from the stent may be optimized for a particular application.

The present invention also provides a method of treating a non-vascular target site by delivering to the non-vascular site a covered non-vascular stent manufactured according to the present invention. The therapeutic agent in the covering of the stent is then allowed to be released into the non-vascular target site to treat the site. Such treatment of the non-vascular target site includes, for example, reduction or inhibition of tumor growth, inflammation, infection, hyperplasia, granulation tissue, or stenosis, or treatment of any other condition that would benefit from localized delivery of a therapeutic agent from a stent.

The foregoing description has been set forth merely to illustrate the invention and is not intended as being limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

1. A method of manufacturing a covering stent comprising:
providing a stent having a distal end and a proximal end and comprising a plurality of segments defining a plurality of openings therebetween;
preparing a viscous mixture comprising a polymer, a solvent, and a therapeutic agent;
applying the viscous mixture to the stent to form a covering on the plurality of segments and the plurality of openings; and
allowing the solvent to evaporate.

2. The method of claim 1, wherein the stent is a non-vascular stent.

3. The method of claim 2, wherein the non-vascular stent is selected from the group consisting of a esophageal stent, biliary stent, pancreatic stent, tracheal stent, lamygeal stent, bronchial stent, prostatic stent, urethral stent, ureteral stent, duodenal stent, and a colonic stent.

4. The method of claim 1, wherein at least one of the proximal end and the distal end are not covered with the covering.

5. The method of claim 1, wherein the polymer is bioresorbable.

6. The method of claim 1, wherein the polymer is silicone, polyurethane or co-polymers thereof.

7. The method of claim 1, wherein the polymer is styrene-isobutylene-styrene.

8. The method of claim 1, wherein the viscous mixture has a viscosity of between about 50 centipoise and about 500 centipoise.

9. The method of claim 1, wherein the polymer percent weight of the viscous mixture is between about 5% and about 25%.

10. The method of claim 9, wherein the polymer percent weight of the viscous mixture is between about 22% and about 23.5%.

11. The method of claim 1, wherein the polymer is silicone and the percent weight silicone of the mixture is between about 20% and about 25%.

12. The method of claim 1, wherein the percent weight of the therapeutic agent in the viscous mixture is between about 0. 1% and about 6%.

13. The method of claim 1, wherein the percent weight of therapeutic agent in the covering after the solvent has evaporated is between about 0.4% and about 50%.

14. The method of claim 1, wherein the therapeutic agent is selected from the group consisting of an antimicrobial agent, antibiotic, anti-inflammatory agent, analgesic agent, anesthetic agent, and anti-cancer agent.

15. The method of claim 1, wherein the step of applying the viscous mixture comprises dipping the stent in the viscous mixture.

16. The method of claim 1, wherein the step of applying the viscous mixture comprises spraying the stent with the viscous mixture.

17. A method of manufacturing a covered stent comprising:
providing a stent comprising a hollow tube having a continuous outer surface;
preparing a viscous mixture comprising a polymer, a solvent, and a therapeutic agent;
applying the viscous mixture to the stent to form a covering on the entire continuous outer surface of the stent; and
allowing the solvent to evaporate.

18. The method of claim 17, wherein the stent is a non-vascular stent.

19. The method of claim 17, wherein the non-vascular stent is selected from the group consisting of a esophageal stent, biliary stent, pancreatic stent, tracheal stent, larnygeal stent, bronchial stent, prostatic stent, urethral stent, ureteral stent, duodenal stent, and a colonic stent.

20. The method of claim 17, wherein the viscous mixture has a viscosity between about 50 centipoise and about 500 centipoise.

21. A method of manufacturing a covered stent comprising:
providing a stent having an outer surface;
preparing a viscous mixture comprising a polymer, a solvent, and a therapeutic agent, the viscous mixture having a viscosity between about 1 10 centipoise and about 190 centipoise;
applying the viscous mixture to the entire outer surface of the stent; and
allowing the solvent to evaporate.

22. The method of claim 21, wherein the stent is a non-vascular stent.

23. The method of claim 22, wherein the non-vascular stent is selected from the group consisting of a esophageal stent, biliary stent, pancreatic stent, tracheal stent, larnygeal stent, bronchial stent, prostatic stent, urethral stent, ureteral stent, a duodenal stent, and a colonic stent.

24. A method of treating a non-vascular target site comprising:
providing a non-vascular stent having an outer surface that is entirely covered with a covering comprising a polymer and a therapeutic agent;
delivering the non-vascular stent to the non-vascular target site; and
allowing the therapeutic agent to be released into the non-vascular target site to treat the non-vascular target site.
